(19) United States
(12) Patent Application Publication
(10) Pub. No.: US 2017/0176549 A1
(43) Pub. Date: Jun. 22, 2017

(54) Use of a Measuring Appliance for Examining Constituents of a Human or Animal Body

(71) Applicant: Robert Bosch GmbH, Stuttgart (DE)

(72) Inventors: Reiner Krapf, Filderstadt (DE); Ulli Hoffmann, Niefern-Oeschelbronn (DE); Andreas Graessl, Leinfelden-Echterdingen (DE); Klaus Marx, Stuttgart (DE)

(21) Appl. No.: 15/383,186

(22) Filed: Dec. 19, 2016

(30) Foreign Application Priority Data
Dec. 21, 2015 (DE) 10 2015 226 168.9

(51) Int. Cl. G01R 33/28; G01R 33/46; A61B 5/055; G01R 33/44

(52) U.S. Cl. CPC G01R 33/28; G01R 33/448; G01R 33/46; A61B 5/055

(57) ABSTRACT

The use of a hand-held, energy-independent measuring appliance comprising a housing, in which provision is made of at least a nuclear magnetic resonance sensor, a control apparatus for controlling the measuring appliance, an evaluation apparatus for evaluating a measurement signal supplied by the nuclear magnetic resonance sensor, an output apparatus for outputting ascertained information and an apparatus for energy supply for the measuring appliance in the form of a battery, in particular a rechargeable battery, for the purposes of examining a constituent of a human or animal body, in particular for examining tissue and/or bodily fluids, preferably for examining blood and/or urine, is proposed.

This application claims priority under 35 U.S.C. §119 to application no. DE 10 2015 226 168.9, filed on Dec. 21, 2015 in Germany, the disclosure of which is incorporated herein by reference in its entirety.

### BACKGROUND

The present disclosure relates to the use of a mobile, hand-held measuring appliance comprising a nuclear magnetic resonance sensor.

DE 10 2014 218 375 A1 and DE 10 2014 218 371 A1 have disclosed mobile measuring appliances, each with a sensor apparatus, wherein the sensor apparatus comprises at least one nuclear magnetic resonance sensor which is provided for determining a humidity value or for detecting and/or analyzing and/or distinguishing between material characteristics of a workpiece to be examined.

### SUMMARY

According to the disclosure, the use of a hand-held, energy-independent measuring appliance comprising a housing, in which provision is made of at least
a nuclear magnetic resonance sensor,
a control apparatus for controlling the measuring appliance,
an evaluation apparatus for evaluating a measurement signal supplied by the nuclear magnetic resonance sensor,
an output apparatus for outputting ascertained information and
an apparatus for energy supply for the measuring appliance in the form of a battery, in particular a rechargeable battery,

for the purposes of examining a constituent of a human or animal body, in particular for examining tissue and/or bodily fluids, preferably for examining blood and/or urine, is proposed.

Here, a “hand-held measuring appliance” should be understood to mean, in particular, that the measuring appliance may be transported without the aid of a transport machine and by using only hands, in particular one hand, and may be guided on and/or along a constituent of a human or animal body to be examined, in particular during a measuring process as well. To this end, the mass of the hand-held measuring appliance is in particular less than 20 kg, advantageously less than 10 kg and particularly advantageously less than 2 kg. In one embodiment, the measuring appliance has a handle or handle region, by means of which the measuring appliance may be guided over a constituent of a human or animal body to be examined. Alternatively, or additionally, for the purposes of the examination thereof, the constituent of a human or animal body to be examined may also be guided to the measuring appliance and/or along the measuring appliance.

In one embodiment of the hand-held measuring appliance, the components of the measuring appliance, in particular the nuclear magnetic resonance sensor, the control apparatus, the evaluation apparatus and the apparatus for energy supply of the measuring appliance, are housed, at least in part, in the housing of the measuring appliance. In particular, the components are housed in the housing of the measuring appliance with more than 50%, preferably with more than 75% and particularly preferably with 100% in terms of the overall volume thereof. In a measuring appliance designed thus, an advantageous use may be realized by easy, in particular one-handed, guidance of the measuring appliance on and/or over a constituent of a human or animal body to be examined. Moreover, the components are thus protected from damage and ambient influences, for example moisture and dust.

The measuring appliance is realized as an energy-independent measuring appliance. “Energy-independent” should be understood to mean that the measuring appliance may be operated independently of a power grid, in particular in a wireless manner, at least on a temporary basis, preferably at least during the duration of an examination of a constituent of a human or animal body. To this end, the measuring appliance comprises an apparatus for energy supply in the form of a power-grid-independent energy store, in particular in the form of a battery, preferably in the form of a rechargeable battery. The apparatus for energy supply is provided to supply the measuring appliance with electric energy for starting up purposes and during the operation. In an alternative embodiment, the power-grid-independent energy store may also be realized as a fuel cell, a capacitor, a hybrid supercapacitor or as any other energy store appearing expedient to a person skilled in the art and/or a combination/plurality thereof. Accumulators with a cell chemistry providing a high power and/or energy density are particularly suitable for supplying the measuring appliance with energy. A high power and/or energy density permits an improved energy supply of the measuring appliance, in particular an energy supply with a longer service life and adapted to a high power requirement of the nuclear magnetic resonance sensor. Currently, these include e.g. accumulators with lithium and lithium ion cell chemistry, in particular lithium iron phosphate accumulators, lithium manganese oxide accumulators, lithium nickel cobalt manganese oxide accumulators, over-lithiated lithium nickel cobalt manganese oxide accumulators, lithium sulfur accumulators, lithium polymer accumulators and lithium oxygen accumulators. In one embodiment, the apparatus for energy supply is realized to be detachable from the measuring appliance by way of an interlocking and/or force-fit connection interface. In this context, “detachable” should be understood to mean, in particular, separable in a non-destructive manner Hence, the apparatus for energy supply is arrangeable on and/or in the measuring appliance, preferably in a removable and interchangeable manner. In the form of such an energy store, the removable apparatus for energy supply may be resupplied and charged with energy from a power grid when within and/or outside of the measuring appliance. In one embodiment of the apparatus for energy supply, the latter is provided also to be usable for supplying energy to other appliances, in particular other measuring appliances and/or other handheld machine tool apparatuses in addition to the use for supplying the measuring appliance with energy.

In particular, “provided” should be understood to mean, specifically, “programmed”, “configured” and/or “equipped”. An object being “provided” for a specific function should be understood to mean, in particular, that the object fulfills and/or carries out this specific function in at least one application and/or operating state, or it is configured to fulfill the function.

For the control thereof, the mobile measuring appliance comprises a control apparatus. The control apparatus has a signaling connection with the other components of the measuring appliance, in particular the nuclear magnetic resonance sensor, the evaluation apparatus, the output apparatus, further e.g. an input apparatus, the apparatus for energy supply and a data communication interface. The control apparatus is provided to communicate with these components during the operation of the measuring appliance. In particular, a “control apparatus” should be understood to mean an apparatus with at least one control electronics element which comprises a means for communication with the other components of the measuring appliance, for example means for open-loop and/or closed-loop control of the nuclear magnetic resonance sensor, means for data processing, means for data storage and/or further means appearing expedient to a person skilled in the art. In one embodiment, the control electronics of the control apparatus should be understood to mean a processor unit in conjunction with a memory unit and an operating program stored in the memory unit, said operating program running during the control procedure. In particular, the electronic components of the control apparatus may be arranged on a circuit board (printed circuit board), for example in the form of a microcontroller.

The measuring appliance has a nuclear magnetic resonance sensor for the purposes of examining a constituent of a human or animal body. The functionality of the nuclear magnetic resonance sensor is based on the nuclear physics effect in which atomic nuclei in the constituent of a human or animal body to be examined absorb and emit alternating electromagnetic fields when in a first magnetic field denoted by B₀. Here, the nuclear magnetic resonance is based on the precession (Larmor precession) of nuclear spins of the atomic nuclei around the magnetic field lines of the first, in particular constant and/or static, magnetic field in the constituent of a human or animal body to be examined. In particular, the nuclear spins of the atomic nuclei in the constituent of a human or animal body to be examined are aligned by the first magnetic field. If energy is radiated onto the atomic nuclei in the form of a second electromagnetic field, in particular in the form an alternating field, for example a pulsed magnetic field, which is in resonance with the Larmor precession of the nuclear spins thereof (energy quanta), the atomic nuclei may change the orientation of the spins relative to the first magnetic field as a result of absorbing this energy. The second magnetic field radiated thereon therefore serves to excite the nuclear spins, which change their nuclear spin states under the reception of energy. Equivalently, the emission of energy quanta subsequently leads to the return of the excited nuclear spins to another, lower energy level and to the emission of an alternating electromagnetic field which may be observed by means of an apparatus for detecting a magnetic field change, in particular by means of an antenna and/or a coil. The atomic nuclei should be understood to mean, in particular, protons (H) and other nuclear magnetic resonance active nuclei such as e.g. 13C, 15N, 19F or 31P.

Therefore, the nuclear magnetic resonance sensor of the measuring appliance allows the excitation of atomic nuclei by means of alternating electromagnetic fields in the constituent of a human or animal body to be examined and the generation of an output signal on account of the nuclear magnetic resonance effect. In particular, excitation of atomic nuclei should be understood to mean that the energy of the electromagnetic fields radiated thereon, in particular alternating fields, causes a change in the nuclear spins of the atomic nuclei. It should be noted that the assumption is made below that, in particular, varying magnetic fields are coupled to electric fields (cf. Maxwell's equations) and so no distinction is made between an electric field and a magnetic field. What is important for the excitation of nuclear magnetic resonance effects is the energy transferred by way of the electromagnetic radiation radiated thereon. In one embodiment, this energy may be transferred by means of pulsed electromagnetic fields.

If the operating parameters of the nuclear magnetic resonance sensor are suitably selected, it is possible to immediately deduce the constituent of a human or animal body and the properties thereof, at least in an examined volume of the constituent of a human or animal body, in the case of the suitable evaluation by means of the measured response signal if the measuring appliance is used. Here, for example, the properties may relate to the composition of the constituent, the presence of substance components and the concentration thereof, or the like.

The “evaluation apparatus” for evaluating at least one measurement signal supplied by the nuclear magnetic resonance sensor should be understood to mean at least one apparatus which comprises an information input for receiving the measurement signals from the nuclear magnetic resonance sensor, an information processing unit for processing, in particular evaluating, the received measurement signals, and an information output for forwarding the processed and/or evaluated measurement signals. In one embodiment, the evaluation unit has components which comprise at least one processor, a memory and an operating program with evaluation and calculation routines. In particular, the electronic components of the evaluation apparatus may be arranged on a circuit board (printed circuit board), in particular on a common circuit board with the control apparatus. In one embodiment, the evaluation apparatus may be realized in the form of a microcontroller. Furthermore, the control apparatus and the evaluation apparatus may be embodied as a single component. The evaluation apparatus is provided to evaluate the measurement signals obtained by the nuclear magnetic resonance sensor and to derive at least information in relation to the examined constituent of a human or animal body therefrom.

The “output apparatus” of the measuring appliance should be understood to mean at least one means which is provided to output at least one varying information item to an operator in an acoustic, optical and/or tactile manner. The evaluation apparatus serves to output to the user of the measuring appliance at least that information which was obtained using the measuring appliance about the examined constituent of a human or animal body. By way of example the output may in this case be realized by means of a display, a touch display, a sound signal, a vibration transducer and/or an LED display. In one embodiment of the output apparatus, the information may be output graphically or alphanumerically as a measurement result of the examination. The output apparatus is housed in the housing of the hand-held measuring appliance. Furthermore, information to be output, for example information about an examined constituent, may also be output to the control apparatus and/or to a data-processing system, in particular for increasing user convenience. The latter comprises at least one information output to an external appliance such as a smartphone, a tablet PC, a PC and any other external data appliance appearing expedient to a person skilled in the art, which external data appliance is connected to the evaluation apparatus of the measuring appliance by way of a data communication interface. Hence, the output apparatus is housed directly in the housing of the measuring appliance and may additionally also be complemented by external output apparatuses. The latter realization option explicitly comprises the control, evaluation and output of the ascertained information by way of wired and/or wireless external systems such as, for example, remote controls, computer controls, tablet PCs and/or other mobile appliances such as cellular telephones, smartphones, etc.

A “constituent of a human or animal body” should be understood to mean any material part of the human body within the biological meaning thereof (in contrast to purely geometrical of physical definitions), i.e. any material part of the body as an organism, independently of whether or not it is alive. Hence, the component of a human or animal body represents a material structure which, in principle, may have different relationships with the human or animal body. By way of example, the constituent may have an anatomical relationship with the structure and/or build of the human or animal body or a physiological relationship with the bodily functions and the metabolic processes being carried out within a body in the process. Furthermore, the constituent may also be related to biochemical processes on a microbiological level. Here, the phrase “constituent of a human or animal body” comprises both liquid and solid constituents. Examples of the constituent of a human or animal body defined thus include arms, legs, head (body parts), skin, tissue samples, nails, bones, brain, bodily fluids such as saliva, blood and/or urine, feces, pus, furuncles, abscesses or the like. Reference is made to the fact that the constituent of a human or animal body to be examined may be situated in or on the living body (organism), in or on the dead body and also outside a human or animal body, in particular in artificial surroundings (e.g. in a test tube).

In particular, “use for examining a constituent of a human or animal body” should be understood to mean the determination of information from the measurement signals obtained from the nuclear magnetic resonance sensor using the measuring appliance and, hence, the derivation of statements relating to multifaceted properties and, in particular, the state of the constituent of a human or animal body. In particular, these statements or this information may relate to the structure, composition, control processes and metabolic processes of the human or animal body, from which it is preferably possible to derive statements in respect of states, diseases, functional disorders, possible convalescence processes or approaches, or the like of the body. In particular, information in respect of substances present in the constituent and the concentration thereof may be ascertained in the case of a suitable evaluation of the measurement signals from the nuclear magnetic resonance sensor. In this way, it is possible, for example, to examine blood and/or urine in respect of inflammation indicators, cancer indicators, kidney values, mineral values, drugs indicators, blood sugar content, oxygen content, cholesterol content, concentrations of low/high-density lipoproteins (LDL, HDL) or other analytes using the measuring appliance. Furthermore, an examination of the vein function (in the context of arteriosclerosis, thrombosis), an examination of bandages in respect of secondary hemorrhaging without opening the bandage, a determination of a blood group, the locating of internal bleeding in organs or an examination of collections of water (edemas) in the body is conceivable.

An examination may be carried out quantitatively in one embodiment, in particular when determining analyte concentrations. Reference is made to the fact that the constituent of a human or animal body to be examined may be examined in or on the living body, in or on the dead body and also outside of a human or animal body, in particular in artificial surroundings (e.g. in a test tube).

From information evaluated thus, an operator of the measuring appliance may easily examine and test a state, a treatment necessity, a treatment capability or the like of the human or animal body in a simple manner. In particular, indicators for medical diagnosis may be determined.

In order to carry out the measurement, the mobile measuring appliance, in particular the nuclear magnetic resonance sensor, is brought into the vicinity of the constituent of a human or animal body to be examined, or vice versa. Here, the use of the measuring appliance allows the examination of a constituent of a human or animal body without impairment of the constituent, i.e., in particular, without destruction, contamination or the like. The nuclear magnetic resonance measuring method is a non-destructive, in particular contactless measuring method, and so a constituent of a human or animal body may be examined without any contact between the measuring appliance and the constituent. In particular, an examination may be carried out in a non-invasive manner using the measuring appliance. Positioning the measuring appliance, in particular the nuclear magnetic resonance sensor contained therein, in the direct vicinity of a constituent of a human or animal body to be examined (or vice versa) facilitates the examination of the constituent of a human or animal body up to a material depth of a few centimeters into the constituent itself. Hence, there may likewise be a subcutaneous examination of a constituent of a human or animal body without destruction (opening up) of the skin.

The hand-held, energy-independent measuring appliance represents a special measuring appliance which, in comparison with scientific nuclear magnetic resonance measuring appliances, has a greatly restricted functionality which is optimized to the examination of a constituent of a human or animal body. In particular, the evaluation apparatus with the evaluation routines thereof is tailored to the examination of constituents of a human or animal body, the evaluation of the information contained therein and the prepared representation and output thereof by means of the output apparatus. When using the measuring appliance for examining a constituent of a human or animal body, the measurement results, i.e. the information relating to the examined constituent of a human or animal body, are prepared for the user of the measuring appliance in an appliance-internal manner immediately after the measurement such that a quick and unique assessment of the examined constituent of a human or animal body is possible in situ, said assessment, in particular, being independent of further appliances such as computers or even of laboratories. Advantageously, a simple and intuitive operation of the measuring appliance which does not require particular previous experience of the operator is achievable.

Moreover, installation size, energy supply and structure of the measuring appliance are adapted in respect of the arrangement of the nuclear magnetic resonance sensor to the mobile, hand-held and energy-independent use of the measuring appliance for examining constituents of a human or animal body. In one embodiment of the measuring appliance, the latter has a planar support surface, against which a constituent of a human or animal body may be placed (or vice versa). In an alternative or additional embodiment of the measuring appliance, the latter has, in the housing, a receptacle for a sample to be examined, in particular for a test tube or the like, and/or a receptacle for a constituent of a human or animal body to be examined, for example for extremities such as a finger. In one embodiment, this receptacle may have a round and/or cylindrical configuration. In an alternative embodiment, this receptacle may also have an open, C-shaped form.

By using the hand-held, energy-independent measuring appliance specifically tailored to the application of examining constituents of a human or animal body for the purposes of examining the constituent, it is possible to realize a precise and comprehensive examination of the constituent of a human or animal body in a quick and non-destructive manner, and hence in a particularly cost-effective manner from an economical point of view. Advantageously, the use according to the disclosure of the hand-held, energy-independent measuring appliance facilitates the quick and precise examination of constituents of a human or animal body in mobile fashion at any location and, in particular, independently of a laboratory.

In one configuration, the measuring appliance is used for examining a constituent of a human or animal body, wherein the measurement signal supplied by the nuclear magnetic resonance sensor, in particular a spectrum and/or a relaxation time of the measurement signal resulting from the excitation of nuclear spins in the constituent of a human or animal body to be examined by way of the nuclear magnetic resonance sensor, is evaluated by means of the evaluation apparatus of the measuring appliance.

According to the disclosure, the mobile measuring appliance is used to comprehensively characterize a constituent of a human or animal body, in particular in view of different features such as composition, concentration of contained substances and/or states of medical interest or the like, and, in particular, to determine indicators for medical diagnosis. Here, spectra and relaxation times may be measured by means of the nuclear magnetic resonance sensor when using the measuring appliance, said spectra and/or relaxation times having a signature or characteristic dependent on the examined constituent, more precisely a signature or characteristic dependent on the atomic structure of the constituent. The evaluation apparatus is specifically embodied for a quick evaluation of the measurement signals supplied by the nuclear magnetic resonance sensor. In particular, quick means within 10 minutes, preferably within 60 seconds, particularly preferably within 5 seconds.

In the case of a suitable selection of the operating parameters of the nuclear magnetic resonance sensor, properties of the constituent of a human or animal body to be examined may be deduced directly by means of a spectrum and/or relaxation times of the response signal. By way of example, in the case of a chemometrics approach, there may be an examination of the constituent of a human or animal body, for example by an evaluation by means of principal component analysis (PCA), without knowing specific cause-effect relationships. Here, the spectrum or a plurality of spectra, chemical shifts, coupling constants, correlations and/or relaxation times or the like are used as input data for the evaluation. As a consequence of the evaluation, point clouds or graphically displayable regions are obtained, which may be evaluated further and, in particular, interpreted in a simple and quick manner by way of a comparison with reference data. By way of example, in one usage form of the measuring appliance, at least one information item from a list of information items may be evaluated, in particular evaluated in a depth-resolved manner, by means of the evaluation apparatus from the measurement signals obtained from the nuclear magnetic resonance sensor, the list comprising at least
a relative and/or absolute hydrocarbon content and/or
bonding states of chemical compounds and/or
a concentration gradient of a material and/or of a compound and/or of an element into the constituent of a human or animal body and/or
time-dynamic processes of chemical compounds and/or
a relative and/or absolute moisture content and/or
further biochemically relevant parameters of the constituent of a human or animal body.

Statements about the bonding states in the constituent of a human or animal body render it possible to determine the material or materials or substances in the constituent of a human or animal body. By way of example, this renders it possible to detect and distinguish different substances, inclusions or the like. It is likewise possible to realize a determination of a material concentration in the examined constituent provided there is a calibration of the nuclear magnetic resonance sensor prior to the measurement. In this manner, it is possible to ascertain water contents, fat contents (HDL, LDL), sugar contents, cholesterol contents, drugs/alcohol contents, mineral contents, allergen contents, toxin contents, or any other contents or concentrations appearing expedient to a person skilled in the art, in a constituent of a human or animal body using the measuring appliance. In one embodiment, a material or substance concentration may be compared to an admissible limit value (threshold) and a warning may be output by the measuring appliance if this threshold is exceeded.

By recording and evaluating time-dynamic processes of chemical compounds, it is possible to examine processes occurring in the constituent of a human or animal body, for example digestion, internal bleeding or the like. Moreover, the measuring appliance may likewise be used to comprehensively characterize a constituent of a human or animal body in respect of moisture. Statements about the relative and/or absolute moisture content and about the moisture gradient in the constituent of a human or animal body facilitate a reliable evaluation of the constituent of a human or animal body, in particular in respect of the presence of gathering water, dehydration or the like.

Further biochemically relevant parameters which may be evaluated using the measuring appliance with the evaluation apparatus comprise e.g. a density and/or porosity of bones in the examined constituent.

Depending on the desired information, the nuclear magnetic resonance sensor measures a spectrum and/or relaxation curves and/or relaxation times, wherein the evaluation apparatus carries out the targeted evaluation of these measurement signals in respect of the desired information.

In one configuration, the measuring appliance is used for examining a constituent of a human or animal body, wherein ascertained information, in particular a spectrum and/or a relaxation time, is compared with reference data from a reference database by means of the evaluation apparatus of the measuring appliance.

In this way, there may be a particularly simple and comprehensive evaluation and/or assessment and/or interpretation of a measured measurement signal from the nuclear magnetic resonance sensor. By way of example, a recorded spectrum and/or recorded relaxation curves, in particular relaxation times, may be compared with reference spectra, reference relaxation curves or reference relaxation times. The comparison of the measurement signals with known reference data which were determined in detail in this case serves for the quick and simple assignment of the measurement signal in order to derive specific information from the measured measurement signal. By way of the comparison with known reference data, it is furthermore possible—in addition to a very high evaluation speed—to obtain a particularly accurate result, i.e. particularly accurate information about the examined constituent of a human or animal body. By way of example, a shift in a measured spectrum may be identified very easily and quickly in a comparison with a reference spectrum, and so information relating to the examined constituent is derivable from this shift.

Advantageously, deviations of the measurement signals from the reference data may be identified in a particularly simple manner by comparing the measured measurement signals with reference data. If these deviations exceed a defined tolerance threshold, they may be interpreted as an indication for irregularity and/or discrepancies in relation to the examined constituent of a human or animal body. In particular, conclusions about possible diseases or anomalies, in particular metabolic diseases, of the human or animal body and/or about diet or the like may be drawn from discrepancies and/or irregularities.

The reference data, in particular reference curves and/or reference values and/or reference spectra and/or reference images, may in this case be stored within the appliance in a database in a memory unit, in particular a memory unit of the control and/or evaluation apparatus. In an alternative or additional embodiment, the reference data may also be stored in a reference database which is external to the appliance and advantageously always up-to-date, for example in a reference database in a computer, a server or a different data memory and/or data processing appliance appearing expedient to a person skilled in the art. In particular, the comparison between the measured measurement signals and the reference data may be carried out by way of an Internet access of the measuring appliance. Alternatively, or additionally, the reference data stored within the appliance may likewise be updated by way of an Internet access of the measuring appliance, for example by comparison with the reference database which is external to the appliance.

In one configuration, the measuring appliance is used for examining a constituent of a human or animal body, wherein ascertained information, in particular a spectrum and/or a relaxation time, and/or a deviation of ascertained information from reference data from a reference database is output, in particular displayed, by means of the output apparatus of the measuring appliance, in particular by means of a display.

Using the information represented by means of the output apparatus, the user of the measuring appliance is able to obtain an intuitively understandable result after carrying out the examination of the constituent of a human or animal body.

In a preferred embodiment, the information relating to the examined constituent is output to the user of the measuring appliance in an intuitively understandable, in particular prepared, manner. In particular, the output is intuitive if the user is able to carry out an examination of a constituent of a human or animal body and subsequently able to derive a statement, preferably an assessment, from the depicted information without prior knowledge. In one embodiment, the preparation of the ascertained information may be carried out e.g. in the form of a color assignment. By way of example, red may, in this case, signal an ascertained critical deviation of an examined target variable—e.g. a concentration—from a prescription—e.g. defined by the reference data in the reference database. By contrast, yellow signals a deviation within an admissible tolerance and green signals an ascertained deviation with an admissible and/or harmless effect. Alternatively, or additionally, an output of the ascertained information may be effected in the form of a short message, which is depicted on the display. By way of example, this short message may contain a specification of the reference data used to evaluate the examined constituent of a human or animal body, further an assessment of the deviation, to the extent that it is ascertained, and recommendations derived therefrom, such as “inject insulin” or “observe a high-magnesium diet” or the like.

In one configuration, the measuring appliance is used for examining a constituent of a human or animal body, wherein the constituent of a human or animal body is examined with spatial resolution, in particular position resolution and/or depth resolution, by means of the measuring appliance.

By way of example, an examination with a lateral spatial resolution may be carried out using a position determination apparatus of the measuring appliance for the purposes of capturing at least one current position of the measuring appliance, in particular in relation to the constituent and/or in relation to the body. The position should likewise be understood to mean an alignment of the measuring appliance, in particular in relation to the constituent and/or in relation to the body. By way of example, the position determination apparatus of the measuring appliance may, in particular, comprise one or more sensors from the group of sensors comprising at least inclination sensors, angle sensors, distance sensors, translation sensors, acceleration sensors and rotational-rate-sensitive sensors. By way of example, the position determination apparatus may be realized using wheels arranged on the housing of the measuring appliance, said wheels recording the change in position when displacing the measuring appliance in relation to the constituent of a human or animal body. In an alternative or additional embodiment, a spatially resolved examination may also be carried out by means of a nuclear magnetic resonance sensor which is swivelable electrically by way of an actuation and/or which is swivelable in a mechanical manner Here, a mechanically swivelable nuclear magnetic resonance sensor constitutes a particularly simple realization of a direction-resolving, and hence spatially resolving, nuclear magnetic resonance sensor. In one embodiment, the mechanically swivelable nuclear magnetic resonance sensor may also be swiveled automatically using an electrical circuit in conjunction with motors or comparable actuators. Furthermore, the nuclear magnetic resonance sensor may also measure in a spatially resolved manner as a consequence of the electrical actuation thereof, for example by way of there being a defined distortion of the first magnetic field by means of gradient coils (shim coils).

A depth-resolved examination of the constituent of a human or animal body may, for example, be carried out by virtue of the nuclear magnetic resonance sensor being shifted or displaced linearly in a mechanical fashion in the depth direction. What can be achieved thus is that the sensitive region characteristic for the nuclear magnetic resonance sensor is shifted into the interior of the constituent of a human or animal body to be examined, and so a depth-resolved measurement may be realized in a simple and particularly economic manner. Alternatively, the sensitive region in the constituent, from which measurement signals are received by means of the nuclear magnetic resonance sensor, may be shifted into the depth—i.e. in the direction into the constituent of a human or animal body to be examined—by way of a change in the frequency with which the alternating electromagnetic field is radiated into the constituent of a human or animal body to be examined.

When using the measuring apparatus for a spatially resolved examination of the constituent of a human or animal body, measurement signals of the nuclear magnetic resonance sensor are evaluated by means of the evaluation apparatus in a manner dependent on the position of the measuring appliance, in particular in relation to the constituent, and/or in a manner dependent on the depth in the constituent of a human or animal body. As a result of this, evaluated information may be correlated with a position of the measuring appliance on the constituent of a human or animal body. Furthermore, multidimensional maps, in which evaluated information is captured in respect of positions of the measuring appliance, in particular in relation to the constituent, may be created by successively repositioning the measuring appliance in relation to the constituent of a human or animal body. Analogously, it is possible to ascertain depth-resolved information from which e.g. profiles in the constituent, i.e. into the constituent, may be gathered. In this way, it is possible, for example, to ascertain moisture curves or concentration curves in the constituent of a human or animal body in one embodiment.

Comprehensive information about the constituent of a human or animal body may be obtained in a particularly effective and efficient manner by way of a spatially resolved, in particular position-resolved and/or depth-resolved, examination of a constituent of a human or animal body using the measuring appliance. By way of example, an examination on the basis of position-dependent variations of the measurement signals supplied by the nuclear magnetic resonance sensor may be carried out by means of a relative measurement or comparison measurement, in which the measuring appliance is moved over the constituent of a human or animal body. Irregularities and/or deviations hidden in the constituent of a human or animal body lead to unique, position-dependent changes in the measurement signals when moving the measuring appliance over the constituent. Information about a substance distribution, for example of a toxin, in the examined constituent of a human or animal body may be ascertained in a simple and quick manner on the basis of such a comparison measurement.

Further, a distribution of a substance may be ascertained particularly well as a consequence of a depth-resolved examination of a constituent in a human or animal body. Here, a gradient of an evaluated property into the constituent of a human or animal body may be ascertained as a consequence of a depth-resolved examination of a constituent of a human or animal body. In one embodiment, it is possible, for example, to determine, in a nondestructive manner, a concentration gradient into the constituent of a human or animal body.

In one configuration, the measuring appliance is used for examining a constituent of a human or animal body, wherein specifications relating to the constituent of a human or animal body to be examined are specified by user inputs by means of an input apparatus and made available to the measuring appliance.

In particular, an input apparatus should be understood to be a means provided to receive at least one information item from a user of the measuring appliance by way of an acoustic, optical, gesture-supported and/or tactile input and forward this to the control apparatus of the measuring appliance. By way of example, the input apparatus may consist of an actuating element, a keyboard, a display, in particular a touch-display, a speech input module, a gesture recognition unit and/or a pointer appliance (e.g. a mouse). Alternatively, or additionally, the input apparatus may also be realized outside of the measuring appliance, for example in the form of an external data processing appliance such as a smartphone, a tablet PC, a PC or the like, which is connected to the control apparatus of the measuring appliance by way of a data communication interface.

By entering specifications in relation to a constituent of a human or animal body, it is possible to advantageously adapt information processing, in particular the evaluation of the measurement signal, the comparison of a measurement signal and/or ascertained information with reference data or the like, to the constituent of a human or animal body to be examined By way of example, a reference database may be selected depending on a user input. Furthermore, it is possible to adapt an operating program of the control apparatus, closed-loop control routines, open-loop control routines, evaluation routines and/or calculation routines, particularly in conjunction with the specification.

By way of example, “specifications in relation to a constituent of a human or animal body” may characterize the constituent itself (“blood”, “urine”, “finger”, “arm”), characterize the body (“male”, “female”, age, weight) and/or further values which are required or expedient for evaluating and/or interpreting the measurement signals. Alternatively, or additionally, further specifications, in particular specifications relating to the physical and/or chemical properties of the constituent of a human or animal body, may be expedient and/or necessary, such as e.g. specifications in relation to “solid or liquid”, “density” or the like.

In one configuration, the measuring appliance is used for examining a constituent of a human or animal body, wherein the measuring appliance is calibrated using a standard sample provided within the appliance, in particular using a tetramethylsilane sample provided within the appliance, before examining the constituent of a human or animal body.

Therefore, for the purposes of examining a constituent of a human or animal body in more detail, there may be a calibration of the measuring appliance, in particular a calibration of the nuclear magnetic resonance sensor, prior to carrying out the examination. In one embodiment, the calibration is carried out using a purely material sample, preferably using a tetramethylsilane (TMS) sample, provided in particular within the appliance, which is used as a standard. All measurements following the calibration, in particular measurements of a constituent of a human or animal body to be examined, are evaluated in relation to this calibration measurement.

Furthermore, a hand-held, energy-independent measuring appliance according to the disclosure for examining a constituent of a human or animal body is proposed, said measuring appliance comprising a housing in which provision is made of at least
a nuclear magnetic resonance sensor,
a control apparatus for controlling the measuring appliance,
an evaluation apparatus for evaluating a measurement signal supplied by the nuclear magnetic resonance sensor,
an output apparatus for outputting ascertained information, and
an apparatus for energy supply in the form of a battery, in particular a rechargeable battery,

wherein the measuring appliance, in particular the nuclear magnetic resonance sensor and/or the evaluation apparatus, is provided for examining a constituent of a human or animal body, in particular for examining tissue and/or bodily fluids, preferably for examining blood and/or urine.

Naturally, the already made descriptions in respect of the use of the measuring appliance, in particular the explanations in respect of the evaluation apparatus and the nuclear magnetic resonance sensor, also apply accordingly to the measuring appliance itself.

In one embodiment of the hand-held measuring appliance, provision is further made of a memory apparatus for storing measurement results and/or working parameters. This memory apparatus may comprise all forms of external and internal electronic memories, in particular digital memories, in particular also memory chips such as USB sticks, memory sticks, memory cards, etc.

Moreover, what is proposed is that the control apparatus and/or the evaluation apparatus of the measuring appliance according to the disclosure comprises a data communication interface for communication, in particular wireless communication, purposes, by means of which the measuring appliance may transmit and/or receive measurement results and/or working parameters. Preferably, the data communication interface uses a standardized communication protocol for transferring electronic data, in particular digital data. Advantageously, the data communication interface comprises a wireless interface, in particular e.g. a WLAN interface, Bluetooth interface, infrared interface, NFC interface, RFID interface, GSM interface or any other wireless interface appearing expedient to a person skilled in the art. Alternatively, the data communication interface may also comprise a wired adapter, for example a USB adapter or micro-USB adapter. Advantageously, measurement results and/or working parameters may, by way of the data communication interface, be transmitted from the measuring appliance to an external data appliance, for example to a smartphone, a tablet PC, a PC, a printer or further external appliances appearing expedient to a person skilled in the art, or said measurement results and/or working parameters may be received by the latter. Advantageously, a transfer of reference data which is usable for further evaluation of measurement signals captured by the measuring appliance may be facilitated by means of the configuration according to the disclosure. In particular, the reference data are in this case recalled from an appliance-internal reference database. Furthermore, multifaceted additional functions may advantageously be facilitated and included, said additional functions, in particular, also requiring direct communication with smartphones (in particular by way of programmed apps) or similar portable data appliances. By way of example, these may comprise automatic mapping functions, firmware updates, data post-processing, data preparation, data comparison with other appliances, etc.

Furthermore, a method according to the disclosure for examining a constituent of a human or animal body, in particular for examining tissue and/or bodily fluids, preferably for examining blood and/or urine, by means of a hand-held, energy-independent measuring appliance is proposed. In a preferred embodiment of the method, the method may, in particular, be characterized by at least the following steps:
i. using an input apparatus, specifying specifications relating to a constituent of a human or animal body to be examined
ii. calibrating the nuclear magnetic resonance sensor using a standard sample, in particular using a tetramethylsilane sample provided within the appliance
iii. placing the measuring appliance against the constituent of a human or animal body to be examined or placing the constituent of a human or animal body to be examined against the measuring appliance
iv. measuring at least one spectrum and/or one relaxation time resulting from the excitation of nuclear spins in the constituent of a human or animal body to be examined
v. evaluating measurement signals from the nuclear magnetic resonance sensor by comparing the measurement signals with reference data from a reference database

outputting the evaluation results, in particular ascertained information and/or a deviation of ascertained information from reference data of a reference database.

### BRIEF DESCRIPTION OF THE DRAWINGS

The disclosure is explained in more detail in the subsequent description on the basis of exemplary embodiments depicted in the drawings. The drawings and the contain numerous features in combination. Expediently, a person skilled in the art will also consider the features on their own and combine these to give further meaningful combinations. In the figures, the same or similar reference signs denote the same or similar elements.

In the drawings:

FIG. 1 shows a perspective illustration of a configuration of the mobile measuring appliance according to the disclosure,

FIG. 2 shows a view of the first housing side of a configuration of the measuring appliance according to the disclosure,

FIG. 3 shows a schematic side view of a configuration of the measuring appliance according to the disclosure,

FIG. 4a shows a schematic sectional illustration of an embodiment of the components forming the nuclear magnetic resonance sensor and the magnetic fields generated thereby,

FIG. 4b shows a schematic sectional illustration of an alternative embodiment of the components forming the nuclear magnetic resonance sensor and the magnetic fields generated thereby, and

FIG. 5 shows a flowchart of an embodiment of the method according to the disclosure.

### DETAILED DESCRIPTION

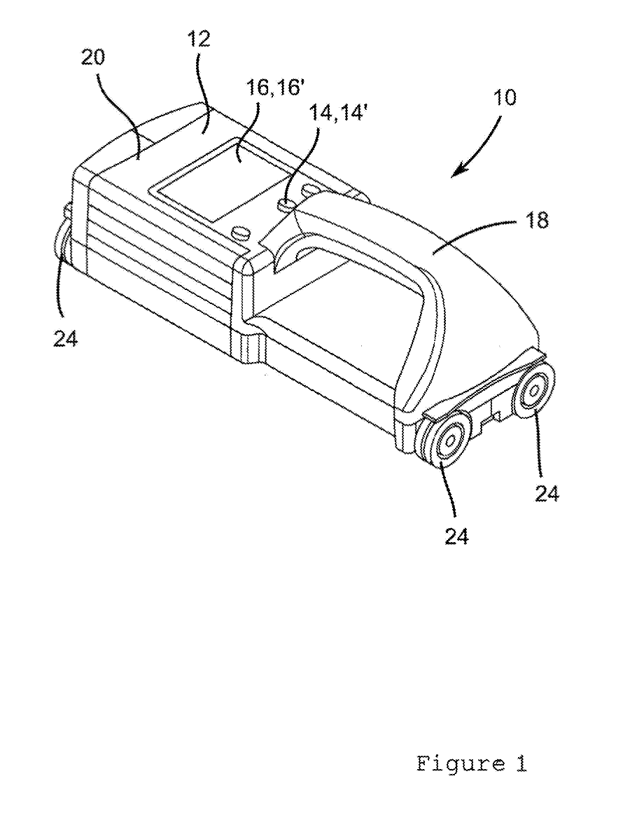

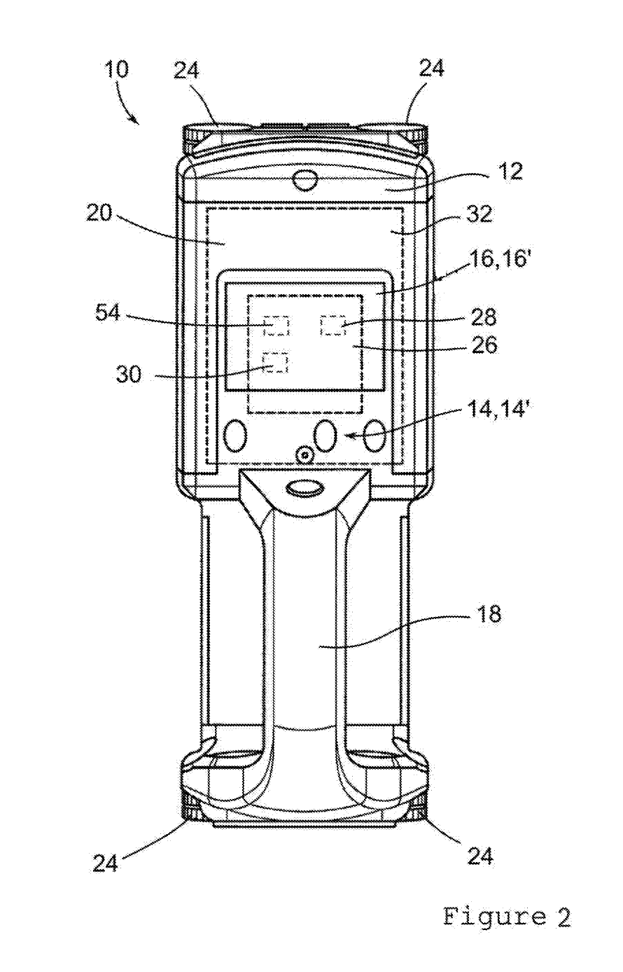

FIG. 1 and FIG. 2 show two views of an exemplary embodiment of the hand-held, energy-independent measuring appliance 10 according to the disclosure, in a perspective illustration and in a simplified, schematic plan view.

The measuring appliance 10 embodied in an exemplary manner comprises a housing 12. The housing 12 houses an input apparatus 14 in the form of actuation elements 14′, suitable for switching the measuring appliance 10 on and off, starting and configuring a measurement process and entering working parameters. Furthermore, an output apparatus 16 in the form of a display 16′, for outputting ascertained information and for outputting working parameters, is provided in the housing 12. For transportation purposes and for the guidance thereof, the measuring appliance 10 comprises a handle 18. The handle 18, the actuation elements 14′ and the display 16′ are situated on a first housing side 20 of the measuring appliance 10 (also referred to as “front side”), which typically faces the user when the measuring appliance is operated.

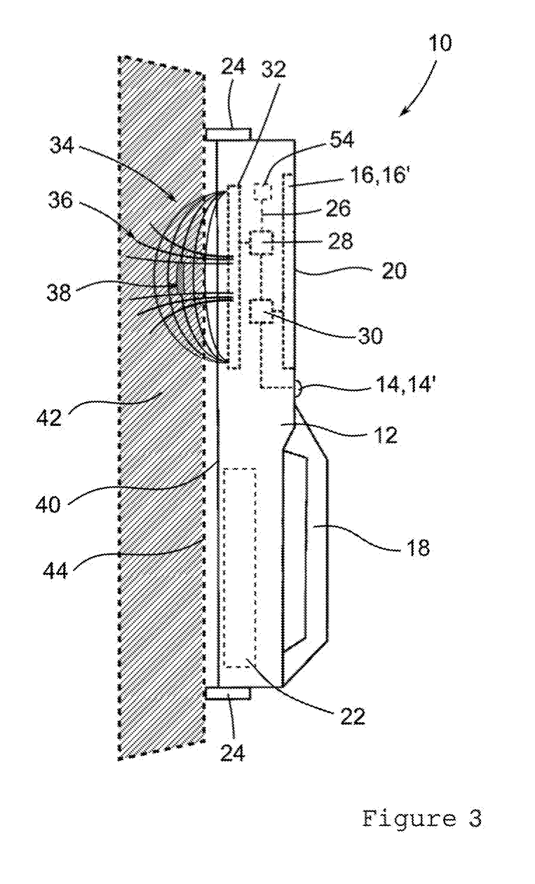

For the purposes of supplying the measuring appliance 10 with energy, the measuring appliance 10 has a recess on the second housing side 40 (also referred to as rear side of the measuring appliance below) lying opposite to the first housing side 20 on the rear side of the appliance, said recess serving to receive power-grid-independent energy stores 22 in the form of rechargeable accumulators (cf. FIG. 3). On account of the power-grid-independent energy store 22, the measuring appliance 10 may be operated, at least temporarily, in an energy-independent manner, i.e. independently of a power grid and hence, in particular, without cables as well. The measuring appliance 10 presented in an exemplary manner comprises lithium ion accumulators, the high energy and power density of which is advantageously suitable for supplying the measuring appliance 10 with energy. In an alternative embodiment, the energy store 22 may also be housed in the handle 18 of the measuring appliance 10. Preferably, the apparatus for energy supply comprises a detachable interlocking and/or force-fit connection interface such that the energy store 22 (in general, also a plurality of energy stores) is (are) arrangeable in a removable and interchangeable manner. Moreover, the energy store 22 may be supplied with energy from a power grid and may be charged within and/or outside of the measuring appliance 10.

The measuring appliance 10 comprises a position determination apparatus in the form of four wheels 24, by means of which the measuring appliance 10 may be displaced on the surface 44 of a constituent 42 of a human or animal body (cf., in particular, FIG. 3). Sensors which are sensitive to rotation of the wheels 24 capture a movement of the measuring appliance 10 and therefore allow measurement results to be related with a position of the measuring appliance 10, in particular in relation to the constituent 42. After placing the hand-held measuring appliance 10 onto the surface 44 of a constituent 42 of a human or animal body to be examined, the change in position of the measuring appliance 10 as a consequence of displacing the measuring appliance 10 on the constituent 42 of a human or animal body is ascertained. These position data are forwarded to an evaluation apparatus 30 for further evaluation.

Further components of the measuring appliance 10, in particular a nuclear magnetic resonance sensor 32, a control apparatus 28 for controlling the measuring appliance 10, an evaluation apparatus 30 for evaluating measurement signals supplied by the nuclear magnetic resonance sensor 32 and a data communication interface 54 connected to the control and/or evaluation apparatus, are housed on a carrier element 26, in particular on a system circuit board or printed circuit board within the housing 12 (see, in particular, FIG. 2).

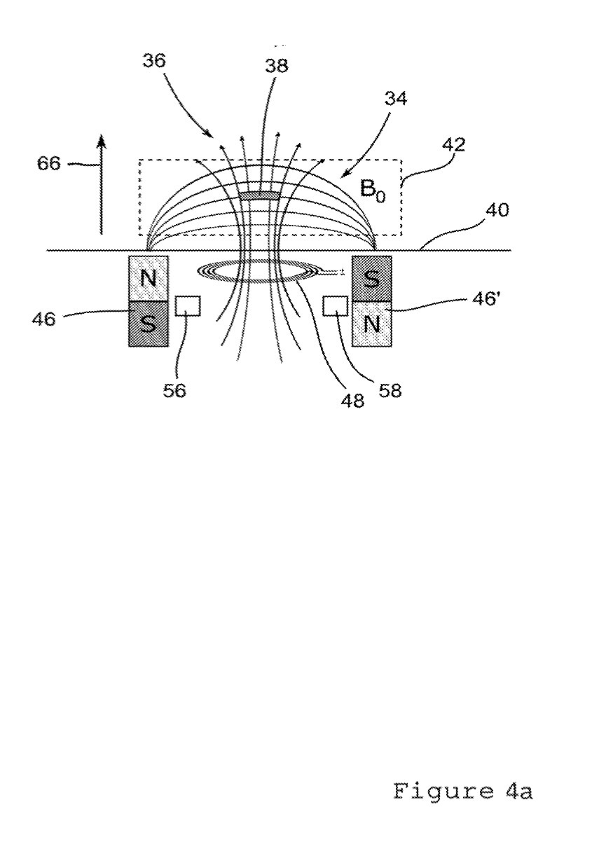

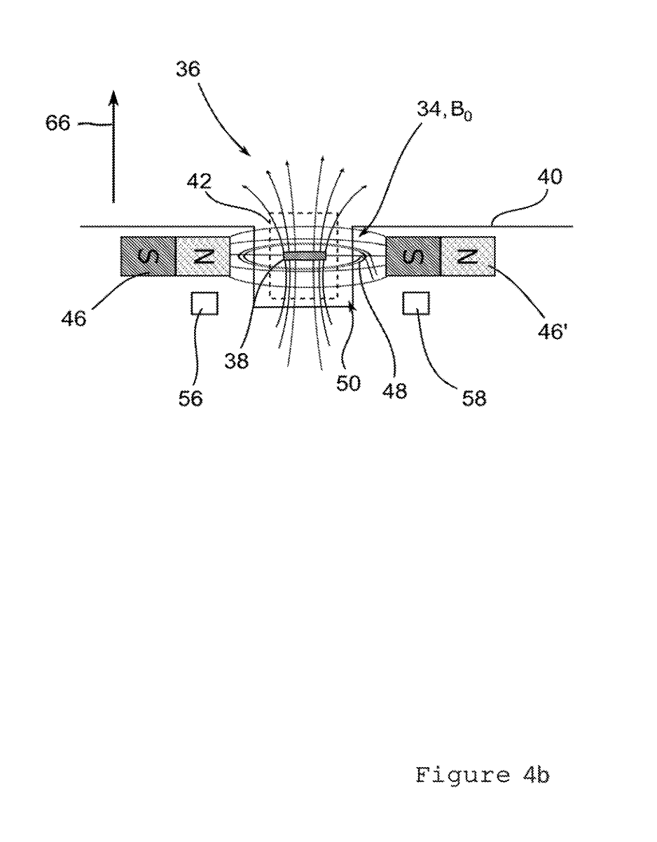

The nuclear magnetic resonance sensor 32, which is explained in detail in the description relating to FIGS. 4a and 4b, is provided to excite the nuclear magnetic resonance in atomic nuclei of the material of the constituent 42 of a human or animal body. According to the disclosure, the measured resonance signal, in particular a spectrum, relaxation curves and/or relaxation times, is used at least for the non-destructive distinction of the constituent 42 of a human or animal body. In this manner, it is possible to ascertain information which, inter alia, relates to the state, a treatment necessity, a treatment capability, a composition of the constituent 42 of a human or animal body, the presence of substance components and the concentration thereof, or the like.

The control apparatus 28 has control electronics comprising means for communicating with the other components of the measuring appliance 10, for example means for open-loop and closed-loop control of the nuclear magnetic resonance sensor 32, of the evaluation apparatus 30 and the like. In particular, the control apparatus 28 comprises a unit with a processor unit, a memory unit and an operating program stored in the memory unit. The control apparatus 28 is provided to adjust at least one operating functional parameter of the measuring appliance 10 depending on at least one input by the user, via the evaluation apparatus 30 and/or via the data communication interface 54.

The evaluation apparatus 30 for evaluating measurement signals supplied by the nuclear magnetic resonance sensor 32 comprises, in particular, an information input, an information processing element and an information output (not depicted in any more detail). Advantageously, the evaluation apparatus 30 consists of at least a processor and a memory with an executable operating program stored thereon, and renders it possible to evaluate at least one measurement signal from the nuclear magnetic resonance sensor 32 and hence determine information relating to the state, a treatment necessity, a treatment capability, a composition of the constituent 42 of a human or animal body, the presence of substance components and the concentration thereof, or the like. Furthermore, the evaluation apparatus 30 has stored correction and/or calibration tables which render it possible to interpret, convert, interpolate and/or extrapolate the evaluation results and calibrate the measuring appliance 10, in particular the evaluation routines, in respect of a constituent 42 of a human or animal body. The evaluation results are output for further use by the evaluation apparatus 30 via the control apparatus 28, either directly to an operator of the measuring appliance 10 or to the data communication interface 54 for the purposes of data transmission. In particular, the evaluation results and/or measurement results may be compared with reference data stored in a reference database using the data communication interface 54.

FIG. 3 depicts the embodiment of the hand-held measuring appliance 10 from FIGS. 1 and 2 in a simplified schematic side view. The nuclear magnetic resonance sensor 32 comprises two apparatuses for generating magnetic fields, in particular a permanent magnet arrangement 46, 46′ (cf. FIGS. 4a, 4b) which generates a first magnetic field 34 (B₀) and a radiofrequency coil 48 (cf. FIGS. 4a, 4b) which generates a second magnetic field 36. The nuclear magnetic resonance sensor 32 is configured in such a way that the first magnetic field 34 is aligned substantially parallel to the second housing side 40 while the second magnetic field 36 is aligned substantially perpendicular to the magnetic field lines of the first magnetic field 34. The two magnetic fields superpose in an extended region, in which the sensitive region 38 of the nuclear magnetic resonance sensor 32 is situated, in particular as a layer-shaped region. The hand-held measuring appliance 10 is positioned with the second housing side 40 in the direct vicinity of a constituent 42 of a human or animal body to be examined, in such a way that the distance between the second housing side 40 and the surface 44 of the constituent 42 of a human or animal body is minimized What this achieves is that the magnetic fields 34, 36 penetrate into the constituent 42 of a human or animal body and the sensitive region 38 comes to rest in the constituent 42 of a human or animal body.

By varying the second magnetic field 36 generated by the second device, i.e., in particular, by varying the radiofrequency coil 48 and/or varying the frequency and/or varying the current and/or varying the voltage in the radiofrequency coil 48, it is possible to vary the sensitive region 38 in terms of its distance from the second housing side 40 (in the direction 66 into the constituent 42 of a human or animal body) and hence modify the distance of the sensitive region 38 in the constituent 42 of a human or animal body from the surface 44 thereof. Alternatively, and/or additionally, the nuclear magnetic resonance sensor 32 may be repositioned in the housing 12 of the measuring appliance 10 in such a way that the distance between the nuclear magnetic resonance sensor 32 and the second housing side 40 is varied and consequently the distance of the sensitive region 38 in the constituent 42 from the surface 44 of the latter is also varied. Depth profiles of the information to be evaluated may be created particularly advantageously in this manner By way of example, it is possible to make a statement about the progress of a decomposition process by way of a depth profile of a moisture curve in a constituent 42 of the human or animal body.

FIG. 4a depicts the nuclear magnetic resonance sensor 32 together with a constituent 42 of a human or animal body to be examined in a schematic sectional illustration of a detail of the exemplary embodiment of the measuring appliance 10 from FIGS. 1 to 3. Two permanent magnets 46, 46′ which are arranged perpendicular to the second housing side 40 and antiparallel in relation to one another generate a first magnetic field 34, in particular a static magnetic field, which extends substantially parallel to the surface of the second housing side 40. This first magnetic field 34 provided for aligning the nuclear spins of the atomic nuclei present in the constituent 42 of a human or animal body has, for example, a magnetic field strength of, in particular, 0.5 Tesla, with the permanent magnets 46, 46′ being produced from a neodymium iron boron alloy. In an alternative embodiment, the magnetic field 34 may also be generated by means of an electromagnet. In this exemplary embodiment, the second apparatus for generating the second magnetic field is formed by a radiofrequency coil 48. As soon as current flows through this coil, an electromagnetic field, in particular the second magnetic field 36, is induced. The two magnetic fields superpose in a region which lies substantially outside of the housing 12 of the measuring appliance 10. The sensitive region 38 of the nuclear magnetic resonance sensor 32 likewise lies in the superposition field of the magnetic fields 34 and 36. Depending on the frequency of the radiated electromagnetic field 36 and the static magnetic field strength of the first magnetic field 34, the sensitive region is defined by an area in an ideal case, in which the magnetic field strength of the first magnetic field 34 is constant and, in particular, has a defined magnitude. In reality, the area in fact has a layered shape on account of non-exact frequencies. Since the magnetic field lines 34 do not extend exactly parallel to the second housing side 40, the sensitive region 38 is therefore curved in a manner corresponding to the magnetic field lines as a consequence thereof. The curvature and form of the first magnetic field 34, and hence of the sensitive region 38, may be influenced and, in particular, homogenized using further means, for example a shim coil 56 and a magnetic shield 58.

The surface 40 of the housing 12 of the measuring appliance 10 represents a plane surface in this exemplary embodiment, on which the constituent 42 of a human or animal body may be placed (or vice versa).

FIG. 4b depicts the nuclear magnetic resonance sensor 32 together with a constituent 42 of a human or animal body to be examined in a schematic sectional illustration of a detail of an alternative embodiment of the measuring appliance 10. Here, the first magnetic field 34, in particular static magnetic field, generated by the first apparatus, in this case two parallel permanent magnets 46, 46′ (with a North-South/North-South sequence) arranged parallel to the second housing side and collinearly, is arranged substantially parallel to a second housing side 40 of the measuring appliance 10 and the second magnetic field 36 generated by the second apparatus, in this case a radiofrequency coil 48, is aligned substantially perpendicular to the first magnetic field 34. A radiofrequency coil 48, the winding plane of which lies collinearly with the direction of extent of the permanent magnets 46, 46′ and parallel to the second housing side 40, lies between the two permanent magnets 46, 46′. This arrangement is positioned in the direct vicinity of the second housing side 40. As soon as current flows through this coil, an electromagnetic field, in particular the second magnetic field 36, is induced. The two magnetic fields superpose in a region which lies substantially outside of the housing 12 of the measuring appliance 10. The sensitive region 38 of the nuclear magnetic resonance sensor 32 likewise lies in the superposition field of the magnetic fields 34 and 36. Depending on the frequency of the radiated electromagnetic field 36 and the static magnetic field strength of the first magnetic field 34, the sensitive region is defined by an area in an ideal case, in which the magnetic field strength of the first magnetic field 34 is constant and, in particular, has a defined magnitude. In reality, the area in fact has a layered shape on account of non-exact frequencies. Since the magnetic field lines 34 do not extend exactly parallel to the second housing side 40, the sensitive region 38 is therefore curved in a manner corresponding to the magnetic field lines as a consequence thereof. The curvature and form of the first magnetic field 34, and hence of the sensitive region 38, may be influenced and, in particular, homogenized using further means, for example a shim coil 56 and a magnetic shield 58.

However, in this exemplary embodiment, the surface 40 of the housing 12 of the measuring appliance 10 does not constitute a plane surface, as in FIGS. 3 and 4a, but has a depression 50 formed specifically for receiving a constituent 42 of a human or animal body to be examined. The depression 50 and the arrangement of the nuclear magnetic resonance sensor 30 are matched to one another in such a way that the constituent 42 of a human or animal body, for example a blood or urine sample situated in a test tube, is introduced into the depression 50. Here, the sensitive region 38 of the nuclear magnetic resonance sensor 32 comes immediately to rest in the constituent 42 of the human or animal body. Furthermore, the depression may also be shaped for receiving, or insertion of, a body part, for example of a finger or a hand.

In one embodiment, the depression 50 is realized as a cylindrically enclosed cavity, with the depression itself being surrounded by cylindrical permanent magnets, for example in a Halbach array. Hence, the permanent magnet surrounds the constituent 42 of a human or animal body inserted into the measuring appliance 10 over the entire length of the circumference thereof. Furthermore, this renders generable a particularly homogeneous first magnetic field 34 (B₀). Alternatively, or additionally, the first magnetic field 34 may also be generated by one or more round coils or round magnets in this exemplary embodiment.

Further embodiments of the measuring appliance 10, in particular of the nuclear magnetic resonance sensor 32 thereof, which are designed for a specific use location and/or a specific use type, are conceivable. By way of example, a U-shaped or C-shaped permanent magnet may be used for measurements on an ear lobe. The less deeply the sensitive region 38 of the nuclear magnetic resonance sensor 32 has to penetrate into a constituent 42 of the human or animal body, the more cost-effective and smaller the appliance can be realized since the required magnetic field strength B₀ reduces drastically with reducing penetration depth of the magnetic field 34.

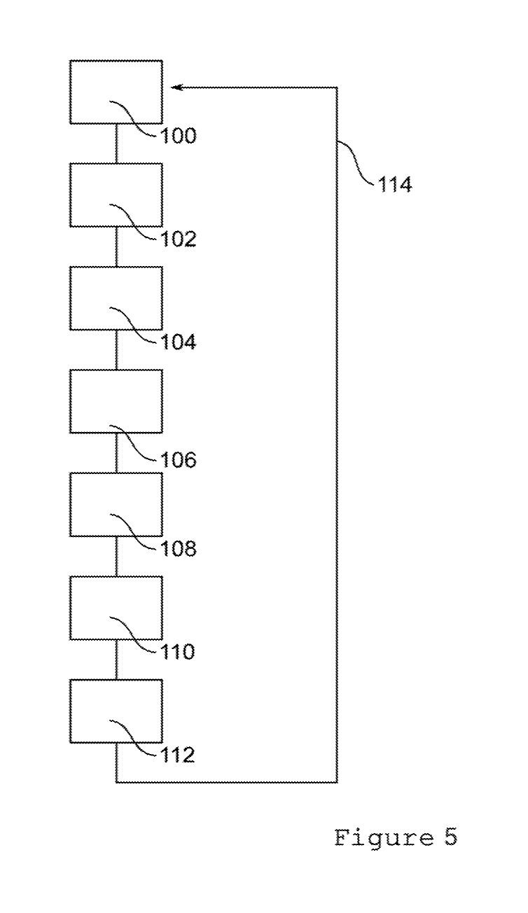

FIG. 5 shows a flowchart which represents an exemplary embodiment of the method according to the disclosure for examining a constituent 42 of a human or animal body by means of a hand-held, energy-independent measuring appliance 10. By way of example, a conceivable application scenario is that of an operator of the measuring appliance 10 haven fallen ill to diabetes and having to carry out a regular blood sugar measurement, from which it is possible to derive whether an insulin injection needs to be carried out (in the case of an elevated glucose concentration in the blood). By using the output apparatus 16, for example by way of a vibration alarm, the measuring appliance 10 sets a reminder for carrying out the measurement.

In a first method step 100, the measuring appliance 10 is then switched on by the operator and it is in an idle mode after a short startup time. Subsequently, specifications in relation to the constituent 42 of a human or animal body to be examined are specified in method step 102 using the input apparatus 14, for example the selection of “finger” and the user profile with specifications relating to the weight and the like in this case. In method step 104, there is a calibration of the nuclear magnetic resonance sensor 32 using a tetramethylsilane sample provided within the appliance, following which the measuring appliance 10 is ready for use for examining the constituent 42 of a human or animal body. For the purposes of measuring a nuclear magnetic resonance signal in the constituent 42, in this case the finger, the finger in method step 106 is introduced into the depression 50 of the measuring appliance 10 in the direct vicinity of the nuclear magnetic resonance sensor 32—in this case in accordance with the geometric design of the measuring appliance 10 according to the exemplary embodiment from FIG. 4b. Here, magnetic fields 34, 36 generated by the nuclear magnetic resonance sensor 32 penetrate out of the measuring appliance 10, through the second housing side 40 and into the finger (constituent 42) of the operator, with the sensitive region 38 coming to rest in the finger (see, in particular, FIG. 4b). Magnetic field changes as a consequence of a nuclear magnetic resonance effect of the nuclear spins of the atomic nuclei excited in the finger, i.e. caused by absorption and/or emission of electromagnetic fields by the atomic nuclei, accompanied by a change in the energy states thereof, are detected by means of the radiofrequency coil 48 of the nuclear magnetic resonance sensor 32 (method step 108). This measurement signal, in particular a measurement signal representing a spectrum and/or relaxation curves, is forwarded to the evaluation apparatus 30, where it is prepared, in particular filtered and/or smoothed, by means of evaluation routines. Subsequently, the measurement signal from the nuclear magnetic resonance sensor is evaluated by comparing the measurement signal with reference data in a reference database (method step 110). In the process, the measured spectra and/or relaxation curves and/or relaxation times are compared to reference spectra or reference relaxation curves or reference relaxation times. Identifying correspondences, and/or deviations, of the measurement signal with, or from, the reference data allows the quick and precise evaluation of the measurement signal in this method step, in which information relating to the examined constituent 42 of the human or animal body, in this case of the finger, are obtained and prepared. The evaluation results, in particular the ascertained information and/or a deviation of the ascertained information from reference data in the reference database, are subsequently forwarded to the output apparatus 16 (method step 112). The evaluated measurement result, i.e. the information relating to the examined constituent 42 of the human or animal body, is depicted to the user on the display 16′ and may additionally be transmitted to a further data processing appliance by way of the data communication interface 54. The output on the display 16′ may be graphical, numerical and/or alphanumerical, for example in the form of a measurement value, a measurement curve, a signal profile, time profile, as image data or in a gradient display, and/or in a combination thereof. Alternatively, or additionally, a representation by means of a signal indicator is possible, in particular by means of e.g. a light-emitting diode, which evaluates a target variable, in this case a blood sugar content and/or the necessity of an insulin injection, by way of a color coding (e.g. red, yellow, green). The method is repeated, in particular in a further examination of the constituent 42 of the human or animal body, indicated by the arrow 114.

In this way, the user may carry out a blood sugar determination non-invasively in this application scenario. Furthermore, predictions relating to the time of the next measurement may be created from a plurality of measurements in quick succession. Furthermore, an installed faulty measurement identification may output a warning if a measurement is, or could be, faulty, for example as a consequence of a finger moving during the measurement (e.g. detected capacitively or by means of a photoelectric sensor).

What is claimed is:

1. A method of examining a constituent of at least one of a human body and an animal body, the method comprising:
examining the constituent of the at least one of the human body and the animal body using a hand-held, energy-independent measuring appliance, the measuring appliance including a (i) housing, (ii) a nuclear magnetic resonance sensor arranged in the housing, (iii) a control apparatus arranged in the housing and configured to control the measuring appliance, (iv) an evaluation apparatus arranged in the housing and configured to evaluate a measurement signal supplied by the nuclear magnetic resonance sensor, (v) an output apparatus arranged in the housing and configured to output ascertained information, and (vi) a rechargeable battery arranged in the housing and configured to supply energy.

2. The method according to claim 1, further comprising:
evaluating, using the evaluation apparatus, at least one of a spectrum and a relaxation time of the measurement signal supplied by the nuclear magnetic resonance sensor resulting from the excitation of nuclear spins in the constituent by way of the nuclear magnetic resonance sensor.

3. The method according to claim 2, further comprising:
comparing, using the evaluation apparatus, the at least one of the spectrum and the relaxation time with reference data from a reference database.

4. The method according to claim 2, further comprising:
displaying, using the output apparatus, at least one of (i) the at least one of the spectrum and the relaxation time and (ii) a deviation of the at least one of the spectrum and the relaxation time from reference data from a reference database.

5. The method of according to claim 1, the examining of the constituent further comprising:
examining, using the measuring appliance, the constituent with spatial resolution.

6. The method of according to claim 1, further comprising:
specifying, with user inputs to an input apparatus that are made available to the measuring appliance, specifications relating to the constituent.

7. The method of according to claim 1, further comprising:
calibrating the measuring appliance, before the examining of the constituent, using a standard sample provided within the measuring appliance.

8. A hand-held, energy-independent measuring appliance, the measuring appliance comprising:
a housing;
a nuclear magnetic resonance sensor arranged in the housing;
a control apparatus arranged in the housing and configured to control the measuring appliance;
an evaluation apparatus arranged in the housing and configured to evaluate a measurement signal supplied by the nuclear magnetic resonance sensor;
an output apparatus arranged in the housing and configured to output ascertained information; and
a rechargeable battery arranged in the housing and configured to supply energy wherein the measuring appliance is configured to examine examining a constituent of at least one of a human body and animal body.

9. Method for examining a constituent of at least one of a human body and animal body using a hand-held, energy-independent measuring appliance, the measuring appliance including a (i) housing, (ii) a nuclear magnetic resonance sensor arranged in the housing, (iii) a control apparatus arranged in the housing and configured to control the measuring appliance, (iv) an evaluation apparatus arranged in the housing and configured to evaluate a measurement signal supplied by the nuclear magnetic resonance sensor, (v) an output apparatus arranged in the housing and configured to output ascertained information, and (vi) a rechargeable battery arranged in the housing and configured to supply energy, the method comprising:
specifying, using an input apparatus, specifications relating to the constituent;
calibrating the nuclear magnetic resonance sensor using a standard sample provided within the appliance;
placing at least one of (i) the measuring appliance against the constituent and (ii) the constituent against the measuring appliance;
measuring at least one of a spectrum and a relaxation time resulting from an excitation of nuclear spins in the constituent;
evaluating measurement signals from the nuclear magnetic resonance sensor by comparing the measurement signals with reference data from a reference database; and
outputting results of the evaluation of the measurement signals.

10. The method according to claim 1, wherein the constituent is at least one of tissue and bodily fluids of the at least one of the human body and the animal body.

11. The method according to claim 1, wherein the constituent is at least one of blood and urine of the at least one of the human body and the animal body.

12. The method according to claim 4, wherein the output apparatus is a display.

13. The method according to claim 5, the examining of the constituent further comprising:
examining, using the measuring appliance, the constituent with at least one of position resolution and depth resolution.

14. The method according to claim 7, wherein the standard sample provided within the measuring appliance is a tetramethylsilane sample.

15. The measuring appliance according to claim 8, wherein the constituent is at least one of tissue and bodily fluids of the at least one of the human body and the animal body.

16. The measuring appliance according to claim 8, wherein the constituent is at least one of blood and urine of the at least one of the human body and the animal body.

17. The method according to claim 9, wherein the constituent is at least one of tissue and bodily fluids of the at least one of the human body and the animal body.

18. The method according to claim 9, wherein the constituent is at least one of blood and urine of the at least one of the human body and the animal body.

19. The method according to claim 9, wherein the standard sample provided within the measuring appliance is a tetramethylsilane sample.

20. The method according to claim 9, the outputting of the results of the evaluation of the measurement signals further comprising:
outputting at least one of (i) ascertained information and (ii) a deviation of the ascertained information from the reference data of the reference database.
